(19) United States
(12) Patent Application Publication
(10) Pub. No.: US 2007/0293543 A1
(43) Pub. Date: Dec. 20, 2007

(54) THRESHOLD BLOOD OMEPRAZOLE CONCENTRATION IS 50 NG/ML FOR THE MAINTENANCE OF INTRAGASTRIC pH OF AT LEAST 4.0 AFTER ORAL DOSING WITH CMA-OMEPRAZOLE, AGN 201904-Z

(76) Inventors: Edward Lee, Los Angeles, CA (US); Dale K. Yu, Silverado, CA (US); John Sefton, Trabuco Canyon, CA (US); Dari D. Parizadeh, Aliso Viejo, CA (US); Diane Tang-Liu, Newport Beach, CA (US)

Correspondence Address: ALLERGAN, INC., 2525 DUPONT DRIVE, T2-7H, IRVINE, CA, 92612-1599, US

(21) Appl. No.: 11/734,691

(22) Filed: Apr. 12, 2007

Related U.S. Application Data
(60) Provisional application No. 60/805,166, filed on Jun. 19, 2006.

(51) Int. Cl. A61K 31/4402; A61P 1/00; C07D 401/12

(52) U.S. Cl. 514/338; 546/273.4

(57) ABSTRACT

Dosage forms of sodium {4-[5-Methoxy-2-(4-methoxy-3,5-dimethyl-pyridin-2-ylmethanesulfinyl)-benzoimidazole-1-sulfonyl]-phenoxy}-acetate, and methods of use of the dosage forms are disclosed herein.

### CROSS-REFERENCE TO RELATED APPLICATION

This application is based on, and claims priority under 35 U.S.C. § 120 to U.S. Provisional Patent Application No. 60/805,166, filed on Jun. 19, 2006, and which is incorporated herein by reference.

### Purpose: To determine the threshold blood omeprazole concentration for the maintenance of intragastric ph of at least 4.0 after oral dosing with AGN 201904-Z, an acid-stable, chemically metered absorption derivative of omeprazole (CMA-omeprazole).

Methods: Forty healthy male subjects received 240, 480, and 640 mg AGN 201904-Z or 40 mg esomeprazole orally once-daily for 5 days in a open-label, randomized, 4-way cross-over study. These doses were estimated to deliver molar equivalent omeprazole doses of 53%, 107%, and 143% of the esomeprazole dose, respectively. Pharmacokinetic (PK) blood samples were collected at 8 timepoints following the 1^{st} and 5^{th} dose of each treatment. Pharmacodynamics (PD) were evaluated via 24-hr intragastric ph monitoring before treatment and after the 1^{st} and 5^{th} doses of each treatment. Empirical PK/PD models were constructed for AGN 201904-Z data by regressing the % time that intragastric pH ≧4.0 during a 24-hr dosing period against the duration of time that blood omeprazole concentrations were greater than 8 different concentrations over the same 24-hr period. The possible threshold concentrations evaluated ranged from 10 to 400 ng/mL. The concentration that provided the best-fit was designated the threshold concentration. The best-fit concentration and model was chosen based on the WSSR, R², visual inspection, and AIC value.
Results: Among the possible omeprazole threshold concentrations evaluated, 50 ng/mL was the best-fit concentration value in all of the models evaluated. The final model suggests the % time intragastric pH ≧4.0 is dependent on the duration of time that blood omeprazole concentrations are maintained above 50 ng/mL. This agreed with the actual results which showed AGN 201904-Z maintained drug concentrations above 50 ng/mL longer than esomeprazole (18.0 vs. 9.2 hr), and also maintained intragastric pH ≧4.0 longer than esomeprazole (80.1 vs. 68.4% of 24-hr; p<0.0001).
Conclusion: The 50 ng/mL blood omeprazole concentration provided the best correlation with the percent time intragastric pH was ≧4.0 over a 24-hr dosing interval and was designated as the threshold concentration of omeprazole AGN 201904-Z, a CMA-omeprazole designed to provide sustained omeprazole exposure, was more effective than esomeprazole at maintaining 24-hour intragastric ph ≧4.0.

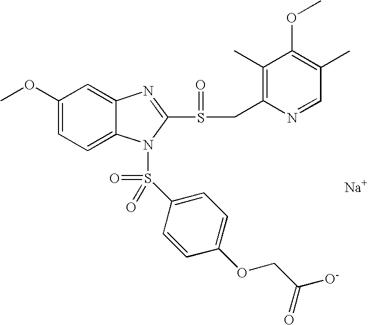

AGN 201904-Z, sodium {4-[5-Methoxy-2-(4-methoxy-3,5-dimethyl-pyridin-2-ylmethanesulfinyl)-benzoimidazole-1-sulfonyl]-phenoxy}-acetate

What is claimed is:

1. A dosage form containing from about 200 to about 700 mg of sodium {4-[5-Methoxy-2-(4-methoxy-3,5-dimethyl-pyridin-2-ylmethanesulfinyl)-benzoimidazole-1-sulfonyl]-phenoxy}-acetate.

2. The dosage form of claim 1 wherein said dosage form contains about 240 mg of sodium {4-[5-Methoxy-2-(4-methoxy-3,5-dimethyl-pyridin-2-ylmethanesulfinyl)-benzoimidazole-1-sulfonyl]-phenoxy}-acetate.

3. The dosage form of claim 1 wherein said dosage form contains about 480 mg of sodium {4-[5-Methoxy-2-(4-methoxy-3,5-dimethyl-pyridin-2-ylmethanesulfinyl)-benzoimidazole-1-sulfonyl]-phenoxy}-acetate.

4. The dosage form of claim 1 wherein said dosage form contains about 640 mg of sodium {4-[5-Methoxy-2-(4-methoxy-3,5-dimethyl-pyridin-2-ylmethanesulfinyl)-benzoimidazole-1-sulfonyl]-phenoxy}-acetate.

5. A method of maintaining stomach pH of a person at or above 4, comprising maintaining the concentration of omeprazole in the blood of a person at or above 50 ng/mL for at least 17 hours during a 24 hour period.

6. The method of claim 5 further comprising orally administering a dosage form of any one of claims 1 to 4 to said person once a day.

7. The method of claim 5 further comprising administering from about 200 to about 700 mg of sodium {4-[5-Methoxy-2-(4-methoxy-3,5-dimethyl-pyridin-2-ylmethanesulfinyl)-benzoimidazole-1-sulfonyl]-phenoxy}-acetate orally to the person once a day.
